(19) United States
(12) Patent Application Publication
(10) Pub. No.: US 2014/0161335 A1
(43) Pub. Date: Jun. 12, 2014

(54) METHOD AND APPARATUS FOR GENERATING SYSTEM RESPONSE OF SCANNER OF IMAGING APPARATUS

(71) Applicant: Samsung Electronics Co., Ltd., Suwon-si (KR)

(72) Inventor: Tae-yong SONG, Hwaseong-si (KR)

(73) Assignee: Samsung Electronics Co., Ltd., Suwon-si (KR)

(21) Appl. No.: 14/046,033

(22) Filed: Oct. 4, 2013

(30) Foreign Application Priority Data
Dec. 7, 2012 (KR) 10-2012-0142316

(51) Int. Cl. A61B 6/00

(52) U.S. Cl. CPC A61B 6/585
USPC 382/131

(57) ABSTRACT

A method and apparatus for generating a system response of a scanner of an imaging apparatus includes generating the system response based on a signal emitted from a point source located in a scanning space of the scanner, setting components that are factors affecting the system response, generating a component response based on a signal received from the scanner with respect to each of the components, and adjusting the system response by using the component responses.

### CROSS-REFERENCE TO RELATED APPLICATIONS

This application claims the benefit of Korean Patent Application No. 10-2012-0142316, filed on Dec. 7, 2012, in the Korean Intellectual Property Office, the entire disclosure of which is incorporated herein by reference for all purposes.

### BACKGROUND

1. Field

The following description relates to a method and apparatus for generating a system response of a scanner of an imaging apparatus.

2. Description of Related Art

A medical imaging apparatus that acquires information about the interior of a human body, in an image of a patient, provides information that is needed for diagnosing diseases of the patient. Medical image scanning methods that are currently used in hospitals or are under development are largely divided into methods of acquiring anatomic images and methods of acquiring physiological images.

First, some examples of scanning technologies that provide detailed anatomic images of a human body at a high resolution include magnetic resonance imaging (MRI) and computed tomography (CT). According to these methods, accurate positions and shapes of organs in a human body are presented by generating a 2-dimensional image of a section of the human body or a 3-dimensional image thereof by using a group of several 2-dimensional images.

Second, positron emission tomography (PET) is a typical physiological image scanning technology that diagnoses malfunctions of a patient's metabolism by scanning metabolic processes in a human body.

In PET, a special radioactive tracer that emits positrons is prepared in the form of a component that may be integrated into the metabolism of a human body. Subsequently, the tracer is injected into the human body by intravenous injection or suction. When a positron emitted from the tracer collides with an electron, two gamma rays of 511 keV are emitted in opposite directions. When the gamma rays are detected by using external equipment, the position of the tracer is tracked and a distribution of tracers and a change of distribution thereof throughout the body of the patient according to time are observed.

### SUMMARY

Provided are a method and an apparatus for generating a system response of a scanner of an imaging apparatus.

Additional aspects will be set forth in part in the description which follows and, in part, will be apparent from the description, or may be learned by practice of the presented embodiments.

In one general aspect, a method of generating an updated scanner response profile of a scanner of an imaging apparatus includes generating a scanner response profile for the scanner based on processing a received signal emitted from a point source located in a scanning space of the scanner, identifying components that are factors that affect the scanner response profile, determining component information based on a signal received from the scanner with respect to each of the components, and updating the scanner response profile based on the component information of the components.

Embodiments may include certain additional features.

In an embodiment, the identifying components include identifying factors indicating an influence by a structural characteristic of the scanner as components.

In an embodiment, the identifying components identifying factors indicating an influence by a physical phenomenon occurring in a process of generating the scanner response profile as components.

In an embodiment, the identifying components include identifying a depth of interaction effect of the scanner is as a component and wherein determining of the component information comprises receiving a signal indicating the depth of interaction effect from the scanner, and the component information is generated based on the received signal.

In an embodiment, the identifying components include identifying a non-collinearity of radioactive rays as a component and wherein the determining of the component information comprises receiving a signal indicating the non-collinearity from the scanner, and the component information is generated based on the received signal.

In an embodiment, the identifying components include identifying a block edge effect of the scanner as a component and wherein the determining of the component information comprises receiving a signal indicating the block edge effect from the scanner, and the component information is generated based on the received signal.

In an embodiment, the identifying components include identifying attenuation by an object located inside the scanner as a component and wherein the determining of the component information comprises receiving a signal indicating the attenuation from the scanner, and the component information is generated based on the received signal.

In an embodiment, the identifying components include identifying a characteristic of the scanner as a component and wherein the determining of the component information comprises receiving a signal indicating the characteristic of the scanner from the scanner, and the component information is generated based on the received signal.

In an embodiment, the identifying components include setting a positron range as the component, and wherein the component information is determined according to the positron range.

In an embodiment, the determining of the component information further comprises determining an overall component information by convoluting the generated component information and updating the scanner response profile comprises applying the overall component information to the scanner response profile.

In another aspect, an apparatus for generating an updated scanner response profile of a scanner of the apparatus includes a scanner response profile generator configured to generate a scanner response profile, based on processing a received signal emitted from a point source located in a scanning space of the scanner, a component identifier configured to identify components that are factors affecting the scanner response profile, a component information determiner configured to determine component information based on a signal received from the scanner with respect to each of the components, and a scanner response profile updater configured to update the scanner response profile based on the component response information of the components.

Embodiments may include certain additional features.

In an embodiment, the component identifier identifies factors indicating a structural characteristic of the scanner as components.

In an embodiment, the component identifier identifies factors indicating an influence by a physical phenomenon occurring in a process of generating the scanner response profile as components.

In an embodiment, the component identifier identifies a depth of interaction effect of the scanner as a component, and the component information determiner receives a signal indicating the depth of interaction effect from the scanner and determines the component information based on the received signal.

In an embodiment, the component identifier identifies non-collinearity of radioactive rays as a component, and the component information determiner receives a signal indicating the non-collinearity from the scanner and determines the component information based on the received signal.

In an embodiment, the component identifier identifies a block edge effect of the scanner as a component, and the component information determiner receives a signal indicating the block edge effect from the scanner and determines the component information based on the received signal.

In an embodiment, the component identifier identifies attenuation by an object located inside the scanner as a component, and the component information determiner receives a signal indicating the attenuation from the scanner and determines the component information based on the received signal.

In an embodiment, the component identifier identifies a characteristic of the scanner as a component, and the component information determiner receives a signal indicating the characteristic of the scanner from the scanner and generates the component information based on the received signal.

In an embodiment, the component identifier identifies a positron range as a component, and the component information determiner determines the component information according to the positron range.

In an embodiment, the component information determiner determines an overall component information by convoluting the determined component information, and the scanner response profile updater updates the scanner response profile by applying the entire component response to the scanner response profile.

In another aspect, a method of generating a scanner response profile of a scanner of an imaging apparatus includes creating an initial scanner response profile for a scanner based on signal information received by the scanner, determining a plurality of factors that interfere with scan quality for the scanner, analyzing the impact of each of the factors on scan quality for the scanner, generating a correction filter that represents a correction to the combined impact of the factors on scan quality, and adjusting the initial profile for the scanner based on the correction filter to provide enhanced scan quality for the scanner.

Embodiments may include certain additional features.

In an embodiment, the signal information is a received signal from a point source located inside the scanner.

In an embodiment, the generation the correction filter comprises convoluting the plurality of factors.

In an embodiment, the factors comprise at least one of a structural characteristic of the scanner, a physical phenomenon occurring in the process of generating the scanner response profile, a depth of interaction effect, a non-collinearity of radioactive rays, a block edge effect of the scanner, attenuation by an object located inside the scanner, or a positron range.

### BRIEF DESCRIPTION OF THE DRAWINGS

FIG. 1 is a diagram illustrating an example of a medical image generating apparatus.

FIG. 2 is a diagram illustrating a view for explaining line-of-response (LOR) data.

FIG. 3 is a diagram illustrating an example when two gamma rays emitted from a tracer do not form a straight line.

FIG. 4 is a block diagram schematically illustrating an example of a system response generation apparatus.

FIG. 5 is a diagram illustrating a view for explaining generation of a component response of a scanner according to a depth of interaction effect.

FIG. 6 is a diagram illustrating a view for explaining generation of a component response of a scanner according to non-collinearity of radioactive rays.

FIG. 7 is a diagram illustrating a view for explaining generation of a component response of a scanner according to a block edge effect.

FIG. 8 is a diagram illustrating a view for explaining generation of a component response of a scanner according to attenuation by an object located inside the scanner.

FIG. 9 is a diagram illustrating a view for explaining generation of a component response of a scanner according to a positron range.

FIG. 10 is a diagram illustrating a view for explaining adjustment of a system response by using an entire component response.

FIG. 11 is a flowchart for illustrating an example method in which the system response generation apparatus of FIG. 4 generates a system response.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

As used herein, the term “and/or” includes any and all combinations of one or more of the associated listed items. Expressions such as “at least one of,” when preceding a list of elements, modify the entire list of elements and do not modify the individual elements of the list.

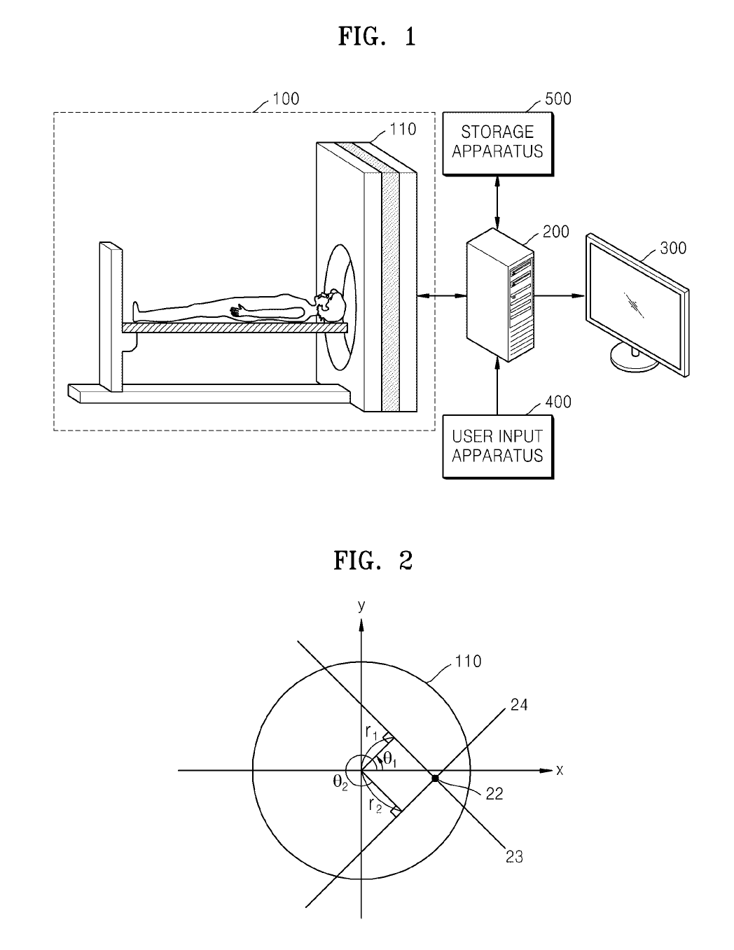

FIG. 1 is a diagram illustrating an example of a medical image generating apparatus. More particularly, FIG. 1 illustrates an overall system for generating an image of a section of a target. Referring to FIG. 1, the medical image generating apparatus includes an imaging apparatus 100, a computer 200, a display apparatus 300, a user input apparatus 400, and a storage apparatus 500.

The medical image generating apparatus of FIG. 1 may generate not only an image of a section of a target, but also a system response of a scanner 110 used to generate a medical image. In an embodiment, the system response indicates a blur model of the scanner 110. The blur model of the scanner 110 is a model used to generate a high resolution image in generating an image by using a signal acquired from the scanner 110 or to correct a low resolution image to a high resolution image. The blur model to correct blur of an image is an example of a system response, which may include other types of information that supplement the image itself by providing information to correct or analyze the image.

Both a method of generating an image of a section of a target by using the medical image generating apparatus of FIG. 1 and a method of generating a blur model of the scanner 110 are described below. In the following description, the term “blur” indicates how wide a point or image spreads. More specifically, when the position of a positron emitting material located in a scanning space in the scanner 110 is estimated by using the scanner 110, the term “blur” is a measure of how much a distribution of estimated positions spreads with respect to the actual position of a positron emitting material. In one example, a point spread function (PSF) is used to indicate the blur.

Furthermore, the medical image generating apparatus may generate a system response, such as a blur model, with respect to the scanner 110 by generating a PSF for each of the positional coordinates by acquiring a signal emitted from a positron emitting material located at each positional coordinate in the scanning space of the scanner 110, and generating a PSF model with respect to the entire scanning space of the scanner 110 by summing the generated PSFs.

In an example of generating an image of a section of a target by using the medical image generating apparatus of FIG. 1, the imaging apparatus 100 detects a signal emitted from a tracer injected into a target. The term tracer refers to a material that emits positrons. For example, the imaging apparatus 100 detects two gamma rays that are emitted as positrons emitted from the positron emitting material injected into a target collide with adjacent electrons. These gamma rays result from the collision, as annihilation occurs when an electron and a positron collide, and the annihilation leads to the emission of two gamma rays. The imaging apparatus 100 transmits line-of-response (LOR) data about the detected gamma rays to the computer 200.

Similar principles apply to the example of generating an image of a section of a patient's body by using the medical image generating apparatus of FIG. 1. Again, the imaging apparatus 100 detects a signal emitted from a quantity of tracer injected into a target. The tracer is a term used to indicate a material emitting positrons. For example, the imaging apparatus 100 detects two gamma rays that are emitted as positrons emitted from the positron emitting material injected into a target's body collide with adjacent electrons, as discussed above. The imaging apparatus 100 transmits LOR data about the detected gamma rays to the computer 200.

In an example of generating a blur model of the scanner 110 by using the medical image generating apparatus of FIG. 1, the imaging apparatus 100 detects two gamma rays that are emitted as positrons emitted from a point source existing somewhere in the scanner 110 collide with adjacent electrons. The imaging apparatus 100 transmits LOR data about the detected gamma rays to the computer 200. The LOR data is data indicating the position of a straight line in a space, as described in detail with reference to FIG. 2.

FIG. 2 illustrates an example of LOR data. Referring to FIG. 2, positrons are emitted from the tracer 22 existing in the scanner 110. As the emitted positrons react with electrons, two gamma rays are emitted in directions separated by 180°. The two gamma rays are placed on one straight line, originating from a common point at which the tracer 22 is located.

FIG. 2 illustrates an example when two straight lines 23 and 24 are detected. Referring to the straight line 23, when a perpendicular line is drawn from the origin of the scanner 110 toward the straight line 23, a distance from the origin to the straight line 23 is “r1” and an angle between an x-axis and the perpendicular line r1 is “θ1”. Thus, the LOR of the straight line 23 is (r1, θ1). Likewise, referring to the straight line 24, when a perpendicular line is drawn from the origin of the scanner 110 toward the straight line 24, a distance from the origin to the straight line 24 is “r2” and an angle between an x-axis and the perpendicular line r2 is “θ2”. Thus, the LOR of the straight line 24 is (r2, θ2). As such, when two or more pieces of LOR data are acquired, the position of a tracer may be determined from the acquired LOR data by applying appropriate geometric techniques. The imaging apparatus 100 transmits the LORs of the detected gamma rays to the computer 200, and the computer 200 may finally determine the position of a tracer based on the received LORs by finding intersections from the LOR data.

Referring back to FIG. 1, the computer 200 generates a medical image of a target by using the data acquired from the imaging apparatus 100. In an example of generating a medical image of a target by using the medical image generating apparatus of FIG. 1, the computer 200 generates a medical image of a section of a target by using the data acquired from the imaging apparatus 100. In an example of generating a blur model of the scanner 110 by using the medical image generating apparatus of FIG. 1, the computer 200 generates a blur model of the scanner 110 by using the data acquired from the imaging apparatus 100.

The display apparatus 300 displays the medical image or blur model generated by the computer 200 on a display panel thereof.

A user may input information needed for operation of the computer 200 by using the user input apparatus 400. For example, a user may issue a command to start or stop the operation of the computer 200 by using the user input apparatus 400.

When the computer 200 generates a medical image of a target, the quality of the medical image is affected by the spatial resolution of the scanner 110. In the case of positron emission tomography (PET), the spatial resolution may be degraded by a non-collinearity of a gamma ray, a positron range, a geometrical structure of a scanner, etc.

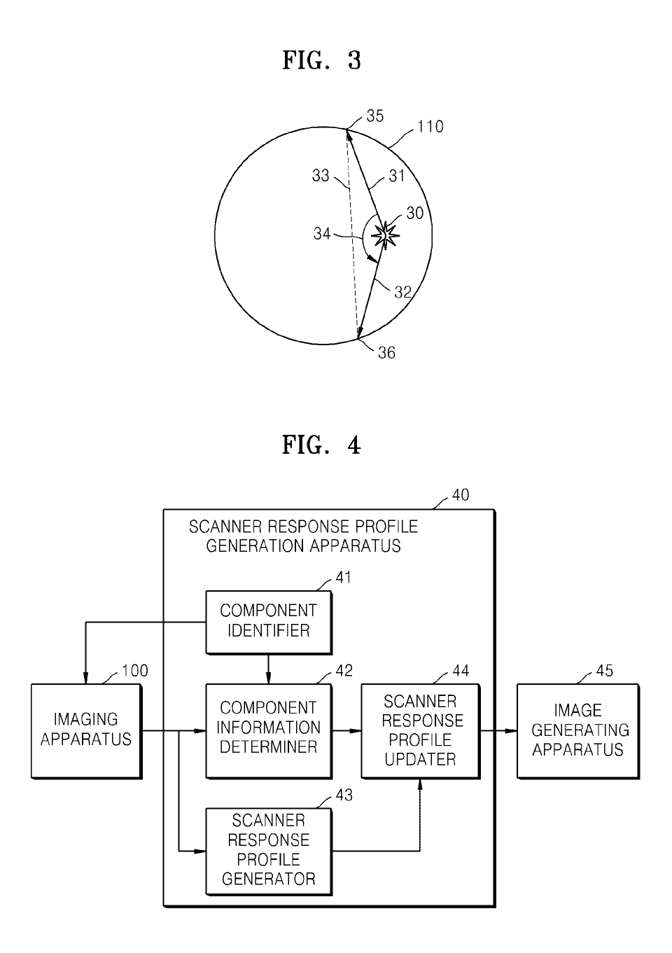

For the non-collinearity of a gamma ray, for example, two gamma rays emitted from a tracer upon the collision of an electron with a positron form an angle that is slightly greater than or less than 180°, not accurately forming 180°, and thus, a resolution of a PET image is degraded. Such a phenomenon is referred to as non-collinearity, an example of which is described below with reference to FIG. 3.

FIG. 3 illustrates an example when two gamma rays emitted from a tracer do not form a straight line. In FIG. 3, two gamma rays 31 and 32 emitted from a tracer 30 form an angle 34 that is slightly less than 180°, and hence there is a discrepancy between angle 34 and 180°. The scanner 110 recognizes positions 35 and 36 where the gamma rays are detected and the tracer is estimated to be located on a straight line 33 connecting the positions 35 and 36. However, the tracer does not actually exist on the straight line 33. Because the tracer is thought to be at the wrong place, the resolution of a PET image suffers. The degradation of resolution of a PET image due to the above discrepancy becomes more remarkable as a diameter of the scanner increases.

The resolution of a PET image is degraded as a positron moves from a tracer before colliding with an electron. For example, after it is emitted from a tracer, a positron loses energy while moving a short distance. The distance the positron moves while losing energy is referred to as a positron range. Then, colliding with an electron, the positron is annihilated emitting a pair of gamma rays having energy of 511 keV. As such, as a positron emits gamma rays after traveling a positron range from a tracer, the position of a tracer and the position where the gamma rays are emitted do not completely match. Thus, when a position where gamma rays are emitted is calculated and the position is assumed to be a position of a tracer, an error occurs. The degradation of resolution of PET due to the above error is referred to as a positron range effect. In general, as the energy of a positron increases, a positron range increases and the resolution of a PET image becomes further degraded.

For a geometrical structure of the scanner 110, for example, resolution is degraded as a distance from the center of the scanner 110 increases due to a time difference according to a difference in depth of interaction for each position caused by the geometrical structure of the scanner 110. For example, a plurality of detecting devices are densely arranged on a surface of the scanner 110. When each scanning device has a rectangular shape that is lengthy in a depth direction, as a gamma ray is obliquely incident on a scanning device, the gamma ray is detected not by one scanning device only but by many adjacent scanning devices at the same time. For this additional reason, tracking an accurate position of a tracer is difficult and thus the resolution of a PET image is degraded.

The resolution of a PET image is degraded by various factors including the above-described factors Since some factors are generated according to the law of probability, there are inherent limits in improving the resolution through technical or mechanical improvement.

Accordingly, to address these resolution issues for PET images, probable blur information corresponding to each voxel of the scanner 110 is generated in the form of a PSF. A blur model of the whole scanner 110 is generated from the generated PSF. The blur model of the scanner 110 is inversely applied to a low resolution PET image captured by the scanner 110 and thus a high resolution image where blur is removed by using the blur model may be generated.

FIG. 4 is a block diagram schematically illustrating an example of a system response generation apparatus 40. Referring to FIG. 4, the system response generation apparatus 40 includes a component setter 41, a component response generator 42, a system response generator 43, and a system response adjuster 44. The system response generation apparatus 40 generates a system response of the scanner 110. A system response of the scanner 110 indicates a characteristic of the scanner. In some embodiments, the system response of the scanner 110 is expressed in the form of a matrix, a function, or data.

The component setter 41 sets components that are factors affecting the system response of the scanner 110. The component setter 41 sets factors indicating a structural characteristic of the scanner 110 and/or factors indicating characteristics of the scanning devices of the scanner 110, as components to affect the system response of the scanner 110.

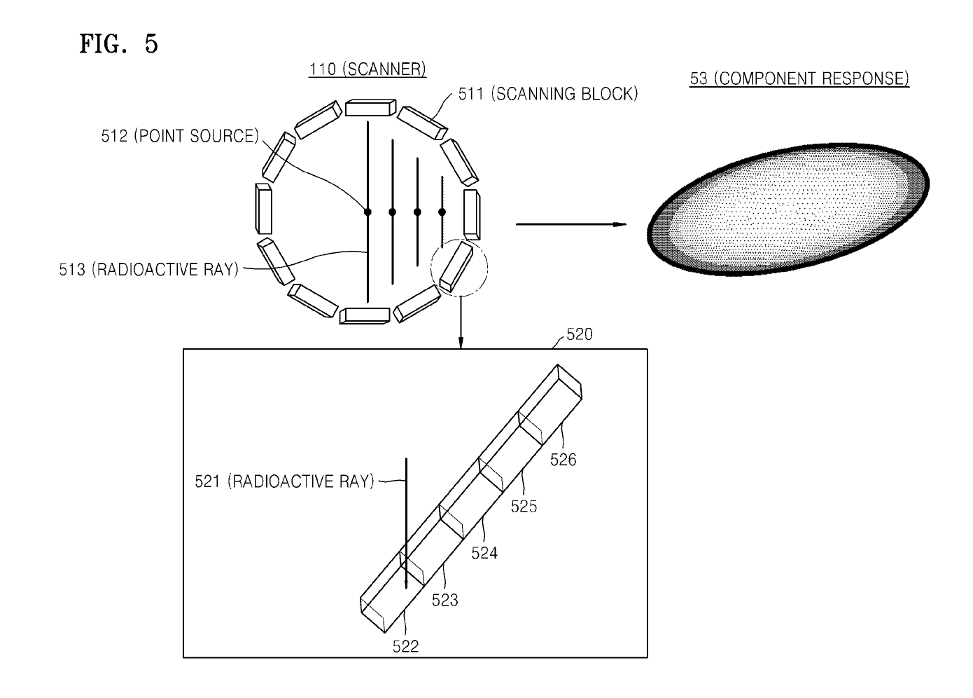

For example, the component setter 41 sets factors indicating an influence by a physical phenomenon occurring in a process of generating a system response, as components. Example factors indicating the structural characteristic of the scanner 110 include a depth of interaction effect, non-collinearity of radioactive rays, a block edge effect, etc. Example factors indicating the characteristic of the scanning devices of the scanner 110 include a detector efficiency, etc. Also, example factors indicating an influence by the physical phenomenon of the scanner 110 include attenuation by an object existing inside the scanner 110, non-collinearity of a radioactive rays, etc. The non-collinearity of radioactive rays is generated by both the structural characteristic and physical attributes of the scanner 110. A detailed description of the role of each component in the non-collinearity will be described in detail with reference to FIGS. 5-9.

The component setter 41 transmits a set component to the imaging apparatus 100 and the component response generator 42. A set component signifies at least one of the above listed components.

The imaging apparatus 100 outputs a signal measured by the scanner 110 with respect to each of the set components to the component response generator 42. In an environment under which the scanner 110 measures a characteristic according to a component, the scanner 110 measures a signal by radioactive rays emitted from the point source and outputs a measured signal to the component response generator 42. A measured signal may be LOR data, as previously discussed.

For example, after a point source or an object is located inside the scanner 110 using the approaches discussed above, the scanner 110 measures a received signal. The position of the point source in the scanner 110 varies according to a component. Efficiency in measuring the position of the point source may be improved by designating the position of the point source by using symmetricity of the scanner 110. The measurement of a signal with respect to a component will be described in detail with reference to FIGS. 5-9.

The component response generator 42 generates a component response with respect to the set component. The component response generator 42 generates a component response based on the signals received from the scanner 110 of the imaging apparatus 100. The component response indicates a characteristic of the scanner 110 with respect to the component and may be a blur model, as discussed. In other words, the component response is a model indicating a blur level generated by the component. In various examples, the component response is generated in the form of a matrix, a function, or data.

The component response generator 42 receives the set component from the component setter 41 and generates a set component response based on a signal received from the imaging apparatus 100. The component response generator 42 generates a component response indicating a difference between a received signal and signals anticipated by the set component. In some embodiments, the component response generator 42 generates a component response indicating an experimentally measured physical phenomenon.

The component response generator 42 generates entire component response by using the generated component responses. The component response generator 42 may generate entire component response by convoluting the generated component responses. The component response generator 42 outputs all generated component responses to the system response adjuster 44.

The system response generator 43 receives a signal from the scanner 110 of the imaging apparatus 100, generates a system response, and outputs a generated system response to the system response adjuster 44.

The system response generator 43 acquires a signal emitted from a point source located in the scanning space of the scanner 110 and generates a system response. The system response generator 43 acquires a signal emitted from a positron emitting material located at each of positional coordinates in the scanning space of the scanner 110 and generates a PSF model with respect to each of the positional coordinates. Then, the system response generator 43 generates the entire scanning space of the scanner 110 by summing all PSFs. Thus, a system response, such as a blur model, with respect to the scanner 110 is generated.

The system response adjuster 44 adjusts a system response by using the component responses. The system response adjuster 44 generates an adjusted system response by applying the component responses to the system response.

The system response generated by the system response generator 43 is generated based on a signal measured by sampling some coordinates of the scanning space of the scanner 110 and locating a point source at each of sampled coordinates only. An estimated value is used as a system response with respect to coordinates that are not sampled.

Thus, in adjusting values of the coordinates that are not sampled, the system response adjuster 44 uses the component response received from the component response generator 42. For example, the system response generator 43 may determine values of coordinates that are not linearly sampled, with respect to coordinates that are located between sampled coordinates and are not sampled. In this case, the values of the coordinates that are not sampled may not have actual linear values. Thus, the system response adjuster 44 adjusts a system response by applying a component response or a blur model to the system response with respect to the values of the coordinates that are not sampled. The adjusted system response has values of the coordinates that are not sampled, which are more accurate than the system response. Thus, using this information improves results by minimizing some of the sources of error previously discussed by correcting for their causes.

An image generating apparatus 45 generates a medical image by applying an adjusted system response to target data. Since the target data includes blur information by the characteristic of the scanner 110, a deblurring process to remove the blur information included in the target data is needed. The image generating apparatus 45 may perform deblurring by inversely applying the adjusted system response of the scanner 110 to the acquired target data. In other words, since the target data includes the target information and the blur information, the blur information included in the target data may be removed by inversely applying the adjusted system response indicating the blur information of the scanner 110 to the target data.

Thus, the image generating apparatus 45 generates a medical image by using the adjusted system response reflecting the characteristic of the scanner 110 so that distortion of a medical image is reduced and the quality of a medical image obtained by the scanner 110 may be improved before generating the final image.

The component setter 41, the component response generator 42, the system response generator 43, and the system response adjuster 44 illustrated in FIG. 4 may correspond to one or a plurality of processors. A processor may be embodied by an array of a plurality of logic gates or by a combination of a general microprocessor and a memory storing a program that is executable in the microprocessor. Also, one of ordinary skill in the art to which the present invention pertains may understand that the above elements may be embodied by other types of hardware.

FIG. 4 illustrates that the system response generation apparatus 40 includes only constituent elements related to the present embodiment. Accordingly, one of ordinary skill in the art to which the present invention pertains may understand that embodiments are not limited to these constituent elements, and other general constituent elements than those illustrated in FIG. 4 may be further included. Additionally, constituent elements illustrated in FIG. 4 may be omitted or replaced appropriately.

FIGS. 5-9 are views for explaining a component response. FIGS. 5-9 illustrate only a portion of the scanner 110. The scanner 110 acquires a signal generated in the scanning space. The scanning space of the scanner 110 corresponds to the inside of a cylinder, as illustrated in FIG. 1. The signal may be a signal emitted from a point source located in the scanning space or from a target into which a tracer is injected.

In a PET apparatus, the signal may be two gamma rays emitted as a positron emitted from a positron emitting material injected into a target's body collides with an adjacent electron.

For a scanner of a PET apparatus, in one example, a plurality of scanning device blocks are arranged on a surface of the scanner and are connected to each other. Also, each scanning device block includes one or more of detecting devices, but in some embodiments each scanning device block includes a plurality of detecting devices. Each scanning device block is separated by a predetermined angle from a neighboring scanning device block. In this example, since an inner surface of each scanning device block is not curved, a section of the scanner may actually have a polygonal shape and not a perfect circular shape.

FIG. 5 is a diagram illustrating a view for explaining generation of a component response of the scanner 110 according to a depth of interaction effect. Referring to FIG. 5, the imaging apparatus 100 measures a signal indicating a depth of interaction effect and outputs a measured signal to the component response generator 42. The signal indicating a depth of interaction effect is measured in an environment where conditions for generating a depth of interaction effect are met.

In an example that the imaging apparatus 100 measures a signal indicating a depth of interaction effect, while a point source is located by being moved from the center of the scanner 110 toward an edge thereof, a signal emitted from a point source 512 is detected. A collimator, not shown, is used to allow radioactive rays 513 emitted from the point source 512 to be emitted in a particular direction or at particular angle. The radioactive rays 513 are emitted in a parallel direction by the collimator, as illustrated in FIG. 5.

Thus, the scanning device on which the radioactive rays 513 are to be incident is previously anticipated and a component response 53 is generated according to whether a signal is output from the anticipated scanning device. In other words, the component response generator 42 may express a blur level to be small when a signal is output from the anticipated scanning device and to be large when a signal is output from a scanning device other than the anticipated scanning device. By estimating blur levels in this manner, the blur levels include information that may be used for image correction.

A block 520 is an enlargement of a scanning block 511. The scanning block 511 includes a plurality of scanning devices 522 to 526. Although FIG. 5 illustrates that a single scanning block includes five scanning devices, the number of scanning devices included in the scanning block 511 is not limited thereto, and may include only one scanning device, and there is no specific maximum of scanning devices that may be included in the scanning block 511.

Referring to the block 520, a depth of interaction effect varies according to transmissivity of a radioactive ray and an angle made by a radioactive ray and a scanning block. As the transmissivity of a radioactive ray increases and the angle between a radioactive ray and a scanning block decreases, the depth of interaction effect becomes large. As the depth of interaction effect is large, a blur level of the scanner 110 increases.

For example, a radioactive ray 521 passes through the scanning devices 522 and 523. Although the radioactive ray 521 is incident on the scanning device 523, the radioactive ray 521 passes through the scanning device 523 and reacts with the scanning device 522. Accordingly, the radioactive ray 521 may react with both of the scanning devices 522 and 523. Also, a scanning device on which the radioactive ray 521 is actually incident and a scanning device having a reaction with the radioactive ray 521 may be different ones. The component response generator 42 generates the component response 53 indicating a difference between the scanning device 522 having a reaction with the radioactive rays 521 and the scanning device 523 on which the radioactive rays 521 is actually incident.

The component response 53 is an example indicating a response according to the depth of interaction effect. For example, the component response 53 indicates a larger blur level at the edge of the scanner 110 than at the center thereof.

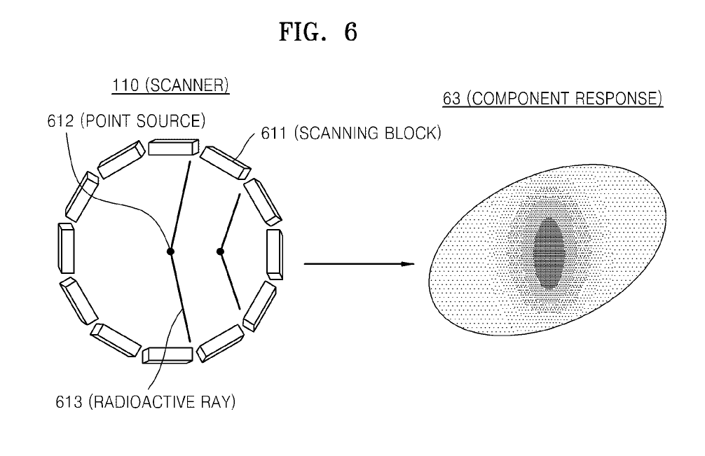

FIG. 6 is a view for explaining generation of a component response of the scanner 110 according to non-collinearity of radioactive rays. Referring to FIG. 6, the imaging apparatus 100 measures a signal indicating non-collinearity of the scanner 110 and outputs a measured signal to the component response generator 42.

In an example, the imaging apparatus 100 measures a signal indicating non-collinearity, while a point source 612 is located by being moved from the center of the scanner 110 to an edge thereof. As this movement occurs, a signal emitted from the point source 612 is detected. A collimator (not shown) is used to allow radioactive rays 613 emitted from the point source 612 to be emitted at particular angle. The radioactive ray 613 is emitted at a particular angle by the collimator, as illustrated in FIG. 6.

The component response generator 42 generates a component response 63 based on a signal output from the scanner 110. In detail, the component response generator 42 generates the component response 53 indicating a difference between a scanning device that is anticipated to detect the radioactive rays 613 and a scanning device that actually detects the radioactive rays 613. The blur level according to the non-collinearity increases as a diameter of the scanner 110 increases. As before, this blur level information may be used to increase image quality.

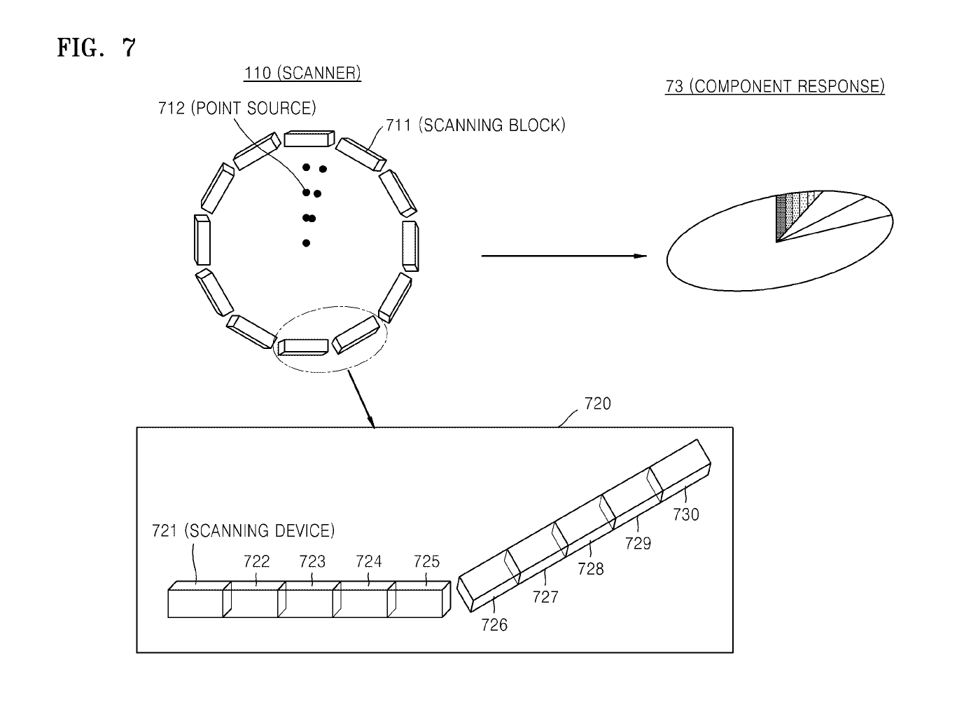

FIG. 7 is a view for explaining generation of a component response of the scanner 110 according to a block edge effect. Referring to FIG. 7, the imaging apparatus 100 measures a signal indicating a block edge effect and outputs a measured signal to the component response generator 42. The block edge effect is generated by the scanner 110 having a polygonal shape, not a circular shape, in an embodiment where several rectangular detectors are used instead of curved detectors, and hence the scanner has a polygonal shape.

In an example, the imaging apparatus 100 measures a signal indicating a block edge effect. While a point source 712 is located by being moved from the center of the scanner 110 to an edge thereof, a signal emitted from the point source 712 is detected. As illustrated in FIG. 7, the imaging apparatus 100 detects a signal emitted from the point source 712 for each case of locating the point source 712 moving toward the center of a scanning block and locating the point source 712 moving toward an edge of the scanning block.

The component response generator 42 generates a component response 73 based on the signal output from the scanner 110. In detail, the component response generator 42 generates the component response indicating a difference between signals detected according to the position of the point source 712.

A region 720 is an enlargement of two scanning blocks 711. A scanning device 725 and a scanning device 726 are arranged with respect to each other at a particular angle. A gap may exist between the scanning device 725 and the scanning device 726 according to a scanner. A radioactive ray emitted from the point source 712 may be detected by a different scanning device due to an angle made by the scanning blocks 711. Also, radioactive ray may not be detected due to the gap existing between the scanning blocks 711. A block edge effect of the scanner 110 varies according to the position of the point source 712. The component response generator 42 generates the component response 73 based on a signal measured according to the position of the point source 712.

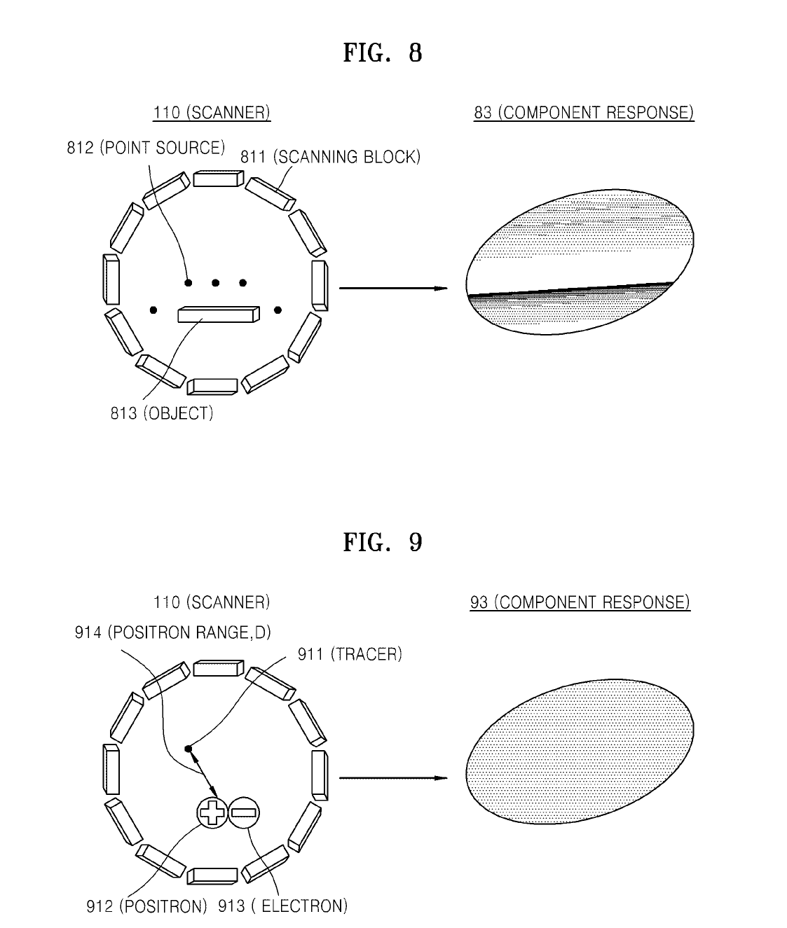

FIG. 8 is a view for explaining generation of a response of the scanner 110 according to attenuation by an object located inside the scanner 110. Referring to FIG. 8, the imaging apparatus 100 measures a signal indicating attenuation by an object (not shown) and outputs a measured signal to the component response generator 42. The attenuation by an object varies according to the size, position, or type of the object located inside the scanner 110.

In an example where the imaging apparatus 100 measures a signal subject to attenuation by an object, the object is located at a particular position in the scanner 110 and a signal emitted from a point source 812 is detected. As illustrated in FIG. 8, the imaging apparatus 100 detects the signal emitted from the point source 812 while changing the position of the point source 812 with respect to the object.

The component response generator 42 generates a component response 83 based on the signal output from the scanner 110. In detail, the component response generator 42 generates the component response 83 indicating a difference between signals detected according to the position of the point source 812.

FIG. 9 is a view for explaining generation of a component response of a scanner according to a positron range 914, D. Referring to FIG. 9, the imaging apparatus 100 outputs what is used as a tracer 911 to the component response generator 42. The positron range 914 of a positron 912 is a distance moved by the positron 912 emitted from the tracer 911 before colliding with an electron 913. The positron range 914 of the positron 912 varies according to the type of the tracer 911 in use. In an example, a value determined through experiments according to the type of the tracer 911 is used as the positron range 914 of the positron 912.

The component response generator 42 generates a component response 93 according to a type of the tracer 911 in use. In detail, the component response generator 42 generates the component response 93 indicating a different blur level according to the tracer 911. In an example, the component response 93 has a constant value over the whole area in the scanner 110.

The imaging apparatus 100 measures a change in scanning efficiency of scanning devices and outputs a measured value to the component response generator 42. The characteristics of the scanning devices change according to time and the scanning devices respectively have different characteristics. Thus, the imaging apparatus 100 measures a change in scanning efficiency of each scanning device and outputs a measured value to the component response generator 42.

The component response generator 42 generates a component response with respect to the scanning efficiency by using a received value. In other words, the component response generates 42 generates a component response indicating the scanning efficiency of each scanning device. The component response generator 42 may store the scanning efficiency of each scanning device of the scanner 100 in the form of a look-up table. For example, the component response generator 42 may generate a component response having a high blur level with respect to a scanning device having a degraded scanning efficiency.

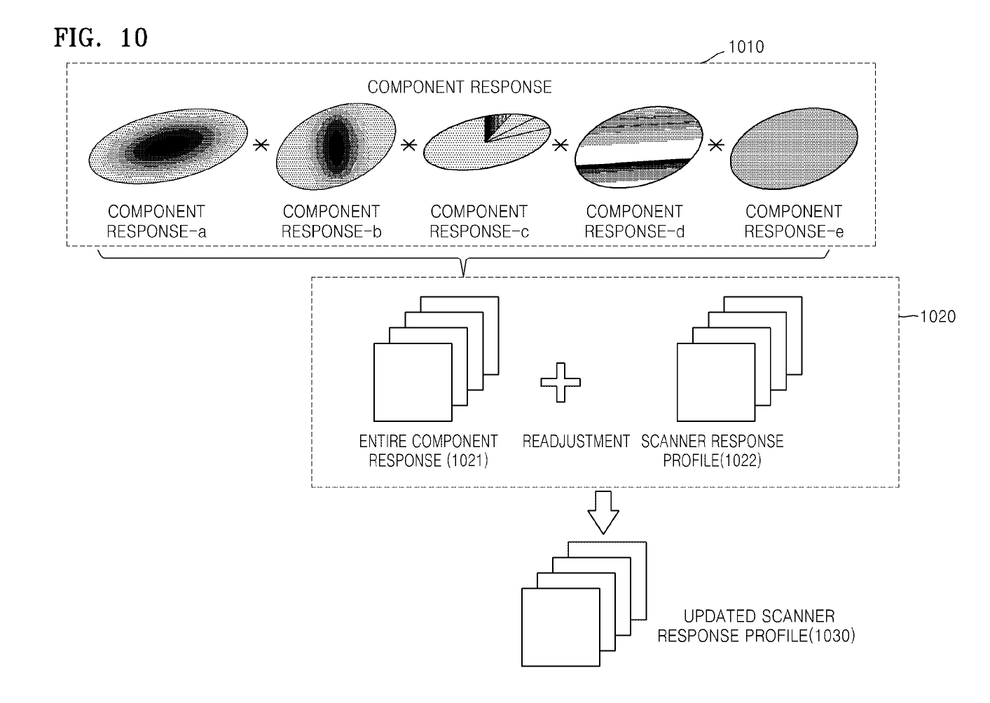

FIG. 10 is a view for explaining adjustment of a system response by using an entire component response. Referring to FIG. 10, a component response 1010 indicates component responses generated by the component response generator 42. In an example, these component responses correspond to the types of component responses discussed with respect to FIGS. 5-9. A block 1020 indicates generation of an adjusted system response 1030 as the system response adjuster 44 applies the entire component response 1021 as an adjustment to a system response 1022.

The entire component response 1012 is generated by convoluting component responses. The component responses “a” through “e” indicate samples of the respective component responses. For example, a component response-a indicates a response of the scanner 110 by a depth of interaction effect, a component response-b indicates a response of the scanner 110 by non-collinearity, a component response-c indicates a response of the scanner 110 by a block edge effect, a component response-d indicates a response of the scanner 110 by attenuation by an object located inside the scanner 110, and a component response-e indicates a response of the scanner 110 by a positron range. These effects have been discussed in depth with respect to FIGS. 5-9. It may also be noted that embodiments may not include component responses for all of these, and that other component response may be included in the entire component response 1021.

As illustrated in FIG. 4, values of coordinates that are not sampled in the system response 1022 may need to be determined. The values of coordinates that are not sampled in the system response 1022 are adjusted and thus the adjusted system response 1030 is generated, taking into account the information provided in the entire component response 1021.

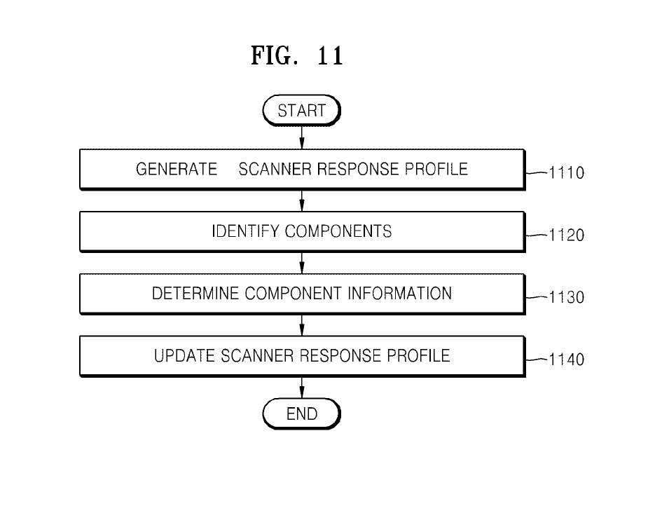

FIG. 11 is a flowchart for illustrating a method in which the system response generation apparatus 40 generates a system response. The method of generating a system response includes operations that are processed in a time series in the system response generation apparatus 40 of FIG. 4. Thus, the above descriptions on the system response generation apparatus 40, though omitted herein, may apply to the method of generating a system response, according to the present embodiment.

In operation 1110, the system response generator 43 generates a system response by acquiring a signal emitted from a point source located in a scanning space of the scanner 110. The scanner 110 acquires a signal emitted from the point source in the scanning space. The signal emitted from the point source is acquired with respect to the entire or partial scanning space of the scanner 110. In one embodiment, when a signal is acquired from a partial space only, a signal with respect to the other part of the scanning space is estimated based on the acquired signal. For example, when the scanner 110 has a cylindrical shape, since there is symmetricity, the signal with respect to the remainder of the scanning space may be estimated by acquiring a signal from a partial space only. The system response generator 43 generates a system response with respect to the entire scanning space of the scanner 110 based on the acquired signal and an estimated signal, if necessary.

In operation 1120, the component setter 41 sets components that are factors affecting a system response. The components may be factors due to physical characteristics of the scanner 110 or a physical phenomenon generated during a process of detecting radioactive rays.

In operation 1130, the component response generator 42 generates component responses based on a signal received from the scanner 110 with respect to each of the set components. The scanner 110 measures signals generated by the set component and outputs a measured signal to the component response generator 42. The component response generator 42 generates a component response indicating a difference between a signal anticipated by the set component and the received signal. In some embodiments, the component response generator 42 generates a component response indicating a physical phenomenon that is experimentally measured.

In operation 1140, the system response adjuster 44 adjusts a system response by using component responses. The system response adjuster 44 receives an entire component response generated by using the component responses. Alternatively, the system response adjuster 44 may receive some of the component responses only from the component response generator 42. When the system response adjuster 44 receives only some of the component responses, the system response adjuster 44 adjusts a system response by using the received component responses only.

The examples of a method and an apparatus described may improve the quality of images obtained as part of medical scanning. By acquiring additional information about errors and image degradation introduced by various factors on the results of scans, embodiments are able to correct for the errors and image degradation that are caused by these factors.

As described above, according to the one or more of the above embodiments of the present inventive concept, an adjusted system response is generated. The response is generated by setting components that are factors affecting a system response of a scanner, generating a response of the scanner for each component, and adjusting the system response of the scanner by using a generated component response. When there is a change in some components, only a response for the changed components is generated and thus a system response is adjusted accordingly.

A hardware component may be, for example, a physical device that physically performs one or more operations, but is not limited thereto. Examples of hardware components include microphones, amplifiers, low-pass filters, high-pass filters, band-pass filters, analog-to-digital converters, digital-to-analog converters, and processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

Software or instructions for controlling a processing device to implement a software component may include a computer program, a piece of code, an instruction, or some combination thereof, for independently or collectively instructing or configuring the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMS, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments for implementing the examples disclosed herein can be easily constructed by a programmer skilled in the art to which the examples pertain based on the drawings and their corresponding descriptions as provided herein.

As a non-exhaustive illustration only, a terminal/device/unit described herein may be a mobile device, such as a cellular phone, a personal digital assistant (PDA), a digital camera, a portable game console, an MP3 player, a portable/personal multimedia player (PMP), a handheld e-book, a portable laptop PC, a global positioning system (GPS) navigation device, a tablet, a sensor, or a stationary device, such as a desktop PC, a high-definition television (HDTV), a DVD player, a Blue-ray player, a set-top box, a home appliance, or any other device known to one of ordinary skill in the art that is capable of wireless communication and/or network communication.

The invention can also be embodied as computer-readable codes on a computer-readable recording medium. The computer-readable recording medium is any data storage device that can store data which can be thereafter read by a computer system. Examples of the computer-readable recording medium include read-only memory (ROM), random-access memory (RAM), CD-ROMs, magnetic tapes, floppy disks, optical data storage devices, etc.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of generating an updated scanner response profile of a scanner of an imaging apparatus, comprising:
generating a scanner response profile for the scanner based on processing a received signal emitted from a point source located in a scanning space of the scanner;
identifying components that are factors that affect the scanner response profile;
determining component information based on a signal received from the scanner with respect to each of the components; and
updating the scanner response profile based on the component information of the components.

2. The method of claim 1, wherein the identifying components comprises identifying factors indicating an influence by a structural characteristic of the scanner as components.

3. The method of claim 1, wherein the identifying components comprises identifying factors indicating an influence by a physical phenomenon occurring in a process of generating the scanner response profile as components.

4. The method of claim 1, wherein the identifying components comprises identifying a depth of interaction effect of the scanner is as a component and wherein determining of the component information comprises receiving a signal indicating the depth of interaction effect from the scanner, and the component information is generated based on the received signal.

5. The method of claim 1, wherein the identifying components comprises identifying a non-collinearity of radioactive rays as a component and wherein the determining of the component information comprises receiving a signal indicating the non-collinearity from the scanner, and the component information is generated based on the received signal.

6. The method of claim 1, wherein the identifying components comprises identifying a block edge effect of the scanner as a component and wherein the determining of the component information comprises receiving a signal indicating the block edge effect from the scanner, and the component information is generated based on the received signal.

7. The method of claim 1, wherein the identifying components comprises identifying attenuation by an object located inside the scanner as a component and wherein the determining of the component information comprises receiving a signal indicating the attenuation from the scanner, and the component information is generated based on the received signal.

8. The method of claim 1, wherein the identifying components comprises identifying a characteristic of the scanner as a component and wherein the determining of the component information comprises receiving a signal indicating the characteristic of the scanner from the scanner, and the component information is generated based on the received signal.

9. The method of claim 1, wherein the identifying components comprises setting a positron range as the component, and wherein the component information is determined according to the positron range.

10. The method of claim 1, wherein the determining of the component information further comprises determining an overall component information by convoluting the generated component information and updating the scanner response profile comprises applying the overall component information to the scanner response profile.

11. An apparatus for generating an updated scanner response profile of a scanner of the apparatus, comprising:
a scanner response profile generator configured to generate a scanner response profile, based on processing a received signal emitted from a point source located in a scanning space of the scanner;
a component identifier configured to identify components that are factors affecting the scanner response profile;
a component information determiner configured to determine component information based on a signal received from the scanner with respect to each of the components; and
a scanner response profile updater configured to update the scanner response profile based on the component response information of the components.

12. The apparatus of claim 11, wherein the component identifier identifies factors indicating a structural characteristic of the scanner as components.

13. The apparatus of claim 11, wherein the component identifier identifies factors indicating an influence by a physical phenomenon occurring in a process of generating the scanner response profile as components.

14. The apparatus of claim 11, wherein the component identifier identifies a depth of interaction effect of the scanner as a component, and the component information determiner receives a signal indicating the depth of interaction effect from the scanner and determines the component information based on the received signal.

15. The apparatus of claim 11, wherein the component identifier identifies non-collinearity of radioactive rays as a component, and the component information determiner receives a signal indicating the non-collinearity from the scanner and determines the component information based on the received signal.

16. The apparatus of claim 11, wherein the component identifier identifies a block edge effect of the scanner as a component, and the component information determiner receives a signal indicating the block edge effect from the scanner and determines the component information based on the received signal.

17. The apparatus of claim 11, wherein the component identifier identifies attenuation by an object located inside the scanner as a component, and the component information determiner receives a signal indicating the attenuation from the scanner and determines the component information based on the received signal.

18. The apparatus of claim 11, wherein the component identifier identifies a characteristic of the scanner as a component, and the component information determiner receives a signal indicating the characteristic of the scanner from the scanner and generates the component information based on the received signal.

19. The apparatus of claim 11, wherein the component identifier identifies a positron range as a component, and the component information determiner determines the component information according to the positron range.

20. The apparatus of claim 11, wherein the component information determiner determines an overall component information by convoluting the determined component information, and the scanner response profile updater updates the scanner response profile by applying the entire component response to the scanner response profile.
